(19) United States
(12) Patent Application Publication
(10) Pub. No.: US 2026/0117168 A1
(43) Pub. Date: Apr. 30, 2026

(54) Method for Synthesizing Nitrogen-Containing Metabolites Using Yeast

(71) Applicant: Meta Organic Technology Inc, Sejong-si (KR)

(72) Inventors: Yi Young Ahn, Gunsan-si (KR); Kyung Hee Han, Seoul (KR); Dong Gi Youn, Seoul (KR)

(21) Appl. No.: 19/366,704

(22) Filed: Oct. 23, 2025

(30) Foreign Application Priority Data
Oct. 24, 2024 (KR) 10-2024-0146821
Oct. 1, 2025 (KR) 10-2025-0143242

(51) Int. Cl. C12N 1/16; C12P 1/02; C12R 1/72

(52) U.S. Cl. CPC C12N 1/165; C12P 1/02; C12R 2001/72

(57) ABSTRACT

A method for producing a nitrogen-containing metabolite using yeast, and a method for synthesizing a metabolite according to an exemplary embodiment of the present disclosure exhibits excellent conversion efficiency by enabling conversion of a precursor substrate into a nitrogen-containing precursor-derived metabolite at a high conversion rate of 90% or higher, and is useful for large-scale production, manufacture, and application.

### CROSS-REFERENCE TO RELATED APPLICATIONS

This application claims priority to Korean Patent Applications No. 10-2024-0146821 filed Oct. 24, 2024, and No. 10-2025-0143242 filed Oct. 1, 2025, the disclosures of which are hereby incorporated by reference in their entireties.

### BACKGROUND

### Technical Field

The following disclosure relates to a method for producing a nitrogen-containing metabolite using yeast, and more particularly, to a method for producing a precursor-derived metabolite by synthesizing the precursor-derived metabolite from a precursor substrate with a high conversion rate through bioconversion using Candida intermedia (Accession number: KCTC16041BP) or a mutant strain thereof.

### TECHNICAL CONSIDERATIONS

The synthesis of metabolites using bioconversion, which is an efficient and environmentally friendly approach, has been widely used in various industrial fields. In particular, a method for synthesizing a nitrogen-containing metabolite using microorganisms has attracted attention as a sustainable technology, as less energy is consumed and fewer toxic by-products are produced compared to conventional chemical synthesis methods. Microorganisms such as yeast, which exhibit high genetic stability and biological safety, are advantageously used in bioconversion processes and may convert various precursor substances to obtain desired products. Such technologies play an important role in the pharmaceutical, agricultural, and chemical industries. Existing art is detailed in Korean Patent No. 10-1915847 (Nov. 6, 2018).

### SUMMARY

A non-limiting embodiment of the present disclosure is directed to providing a method for synthesizing a nitrogen-containing metabolite by converting various precursors, specifically, substances such as saponins, using yeast that is a type of yeast having high genetic stability and biological safety, and that is widely used in bioconversion processes as described above, the yeast being directly isolated by the applicant.

In one non-limiting aspect, a method for producing a nitrogen-containing metabolite includes: culturing Candida intermedia (Accession number: KCTC16041BP) or a mutant strain thereof to obtain a culture broth; preparing a mixed medium containing one or two or more carbon sources selected from the group consisting of maltose, glucose, poly(xylose), fructose, lactose, sucrose, and soluble starch, and one or two or more nitrogen sources selected from the group consisting of ammonium sulfate, arginine, glutamine, and asparagine; and adding the culture broth and a precursor substrate to the mixed medium and performing culture.

In an exemplary embodiment of the present disclosure, the mixed medium may further contain one or two or more catalytic factors selected from the group consisting of calcium nitrate tetrahydrate, magnesium nitrate hexahydrate, magnesium sulfate, monopotassium phosphate, and monosodium phosphate.

In an exemplary embodiment of the present disclosure, the catalytic factor may be contained in the mixed medium at a concentration of 0.05 g/L to 0.15 g/L.

In an exemplary embodiment of the present disclosure, the precursor substrate may include saponin.

In an exemplary embodiment of the present disclosure, the saponin may include one or two or more selected from the group consisting of ginsenoside Re, ginsenoside Rf, ginsenoside Rg1, ginsenoside Rh1, ginsenoside Rg3, ginsenoside Rh2, and ginsenoside Rb2.

In an exemplary embodiment of the present disclosure, the precursor substrate may be contained in the mixed medium at a concentration of 0.5 g/L to 1.5 g/L.

In an exemplary embodiment of the present disclosure, the carbon source may be contained in the mixed medium at a concentration of 15 g/L to 25 g/L.

In an exemplary embodiment of the present disclosure, the nitrogen source may be contained in the mixed medium at a concentration of 2.5 g/L to 7.5 g/L.

In another non-limiting aspect, a method for isolating and purifying a nitrogen-containing metabolite includes isolating and purifying a transformed culture broth obtained according to the method for producing a nitrogen-containing metabolite.

In an exemplary embodiment of the present disclosure, the isolating and purifying may be performed by centrifugation, extraction, filtration, or chromatography.

Other features and aspects will be apparent from the following detailed description, the drawings, and the claims.

### DETAILED DESCRIPTION

Hereinafter, a method for synthesizing a nitrogen-containing metabolite using yeast according to the present disclosure will be described in detail with reference to the accompanying tables or drawings.

When drawings are described, the drawings are provided by way of example to ensure that the concept of the present disclosure may be fully understood by those skilled in the art. Therefore, the present disclosure may be implemented in different forms without being limited to the drawings to be described below, and the drawings may be exaggerated in order to specify the concept of the present disclosure.

The terms “first”, “second”, and the like may be used to describe various components, but the components should not be limited by these terms. The terms are merely used to distinguish one component from another component. For example, without departing from the scope of the present disclosure, a first component may be referred to as a second component, and similarly, the second component may be referred to as the first component.

In this case, unless otherwise defined, the technical terms and scientific terms used herein have the same meanings as commonly understood by those skilled in the art to which the present disclosure pertains. The description for the known function and configuration unnecessarily obscuring the gist of the present disclosure will be omitted in the following description and the accompanying drawings. Terms that are defined in commonly used dictionaries are to be interpreted as having meanings consistent with the contextual meaning in the relevant technical field, and, unless explicitly defined in the present application, should not be interpreted as having idealized or overly formal meanings.

In addition, unless the context clearly indicates otherwise, singular forms used in the specification of the present disclosure may be intended to include plural forms.

In addition, unless otherwise specified, the units used in the specification of the present disclosure are based on weight, and, for example, a unit of % or a ratio refers to wt % or a weight ratio.

In addition, the expression “comprise(s)” as used in the specification of the present disclosure is intended to be an open-ended transitional phrase having an equivalent meaning to “include(s)”, “contain(s)”, “have (has)”, and “are (is) characterized by”, and does not exclude elements, materials, or steps, all of which are not further recited herein. In addition, the expression “substantially consists of” means that other elements, materials, or processes not listed together with the specified elements, materials, or processes may be present in an amount that does not materially affect at least one fundamental and novel technical feature of the present disclosure. In addition, the expression “consists of” means that only the elements, materials, or processes recited are present.

As used herein, the term “and/or” includes any and all possible combinations of one or more of the associated listed items, and also includes the absence of any combination when interpreted in the alternative as “or”.

The term “substantially” or “essentially” means almost entirely or completely, for example, 95% or more of a given amount. In some exemplary embodiments, the term “substantially” or “essentially” means 95%, 96%, 97%, 98%, 99%, 99.5%, or 99.9%.

When used to describe a selection of any component, range, dosage form, or the like disclosed herein, the term “allowable”, “effective”, or “sufficient” means that the component, range, or dosage form is suitable for the disclosed purpose.

In some exemplary embodiments, the terms “first”, “second”, “third”, “fourth”, or similar terms in component names are used to distinguish and identify more than one component that share a certain identity in their names. For example, the terms “first culture broth” and “second culture broth” are used to distinguish between the two culture broths.

Hereinafter, a method for producing a nitrogen-containing metabolite according to the present disclosure will be described in detail.

The present disclosure relates to a method for producing a nitrogen-containing metabolite, the method including: culturing Candida intermedia (Accession number: KCTC16041BP) or a mutant strain thereof to obtain a culture broth; preparing a mixed medium containing one or two or more carbon sources selected from the group consisting of maltose, glucose, poly(xylose), fructose, lactose, sucrose, and soluble starch, and one or two or more nitrogen sources selected from the group consisting of ammonium sulfate, arginine, glutamine, and asparagine; and adding the culture broth and a precursor substrate to the mixed medium and performing culture.

Candida intermedia (Accession number: KCTC16041BP) is a yeast strain isolated and deposited by the applicant. Although the reason is not clearly understood, this strain exhibited superior metabolite conversion efficiency compared with other yeast species or yeast lineages such as Saccharomyces cerevisiae, Saccharomyces ellipsoideus, Saccharomyces bayanus, Schizosaccharomyceses, Tremellomycetes, and Microbotryomycetes.

The mixed medium is not particularly limited, but may further contain a catalytic factor. For example, the mixed medium may further contain one or two or more selected from the group consisting of calcium nitrate tetrahydrate, magnesium nitrate hexahydrate, magnesium sulfate, monopotassium phosphate, and monosodium phosphate, and preferably, calcium nitrate tetrahydrate, which may achieve a remarkable increase in the conversion rate of the precursor substance during the bioconversion process using yeast.

A concentration of the catalytic factor contained in the mixed medium is not particularly limited, but may be, for example, 0.01 g/L to 0.25 g/L, preferably 0.02 g/L to 0.20 g/L, and more preferably 0.05 g/L to 0.15 g/L. Within these ranges, a remarkable increase in the conversion rate of the precursor substance may be achieved, which is therefore preferable.

The precursor substrate is not particularly limited as long as it is a substance to be converted through yeast culture, but may be, for example, saponin. Specific examples of the saponin include one or two or more selected from the group consisting of ginsenoside Re, ginsenoside Rf, ginsenoside Rg1, ginsenoside Rh1, ginsenoside Rg3, ginsenoside Rh2, and ginsenoside Rb2, and, more specifically, ginsenoside Re may be exemplified. When a bioconversion was performed using Candida intermedia (Accession number: KCTC16041BP) that was isolated and identified in the present disclosure according to the synthesis method of the present disclosure, a high conversion rate of 90% or higher was achieved.

A concentration of the precursor substrate contained in the mixed medium is not particularly limited, but may be, for example, 0.1 g/L to 2.5 g/L, preferably 0.3 g/L to 2.0 g/L, and more preferably 0.5 g/L to 1.5 g/L. Within these ranges, a remarkable increase in the conversion rate of the precursor substance may be achieved, which is therefore preferable.

A concentration of the carbon source contained in the mixed medium is not particularly limited, but may be, for example, 5 g/L to 35 g/L, preferably 10 g/L to 30 g/L, and more preferably 15 g/L to 25 g/L. Within these ranges, a remarkable increase in the conversion rate of the precursor substance may be achieved, which is therefore preferable.

A concentration of the nitrogen source contained in the mixed medium is not particularly limited, but may be, for example, 1.0 g/L to 9.0 g/L, preferably 2.0 g/L to 8.0 g/L, and more preferably 2.5 g/L to 7.5 g/L. Within these ranges, a remarkable increase in the conversion rate of the precursor substance may be achieved, which is therefore preferable.

Hereinafter, a method for isolating and purifying a nitrogen-containing metabolite according to the present disclosure will be described in detail.

In addition, the present disclosure relates to a method for isolating and purifying a nitrogen-containing metabolite, the method including isolating and purifying a target metabolite from a biologically converted culture broth obtained according to the production method described above.

The method for isolating the biologically converted metabolite is not particularly limited, any isolation and purification method conventionally known in the art may be used without limitation, and examples thereof include one or two or more selected from the group consisting of an adsorption-membrane separation combined process, filtration, chromatography, membrane separation, recrystallization, and distillation. Specific examples thereof include, but are not limited to, techniques for isolating proteins with high purity through multi-step chromatography processes. By appropriately arranging and applying such isolation and purification methods, the biologically converted metabolite may be harvested on a large scale with minimal by-products.

The exemplary embodiments of the present disclosure described above may be applied equally to each other as long as they are not mutually inconsistent, and modifications that are appropriately made and implemented by those skilled in the art are also included within the scope of the present disclosure.

Hereinafter, the present disclosure will be described in more detail with reference to examples. The examples are provided merely for illustrating the present disclosure in more detail and are not intended to limit the scope of the present disclosure.

### [Reagents, Materials, and Experimental Protocols]

All reagents and materials used in the present disclosure were purchased from well-known reagent suppliers such as Sigma-Aldrich (MO, USA), unless otherwise specified.

### [Example 1] Production of Nitrogen-Modified Metabolite Using Yeast

### 1-1. Yeast Culture

Candida intermedia (Accession number: KCTC16041BP) was used as the yeast strain.

First, arginine was added to a YPD medium as a nitrogen source so that a concentration was 5.0 g/L, and then, the mixture was stirred at 150 rpm for 12 hours. After inoculating the yeast strain, the culture was placed in a constant-temperature and constant-humidity incubator, cultured at 27.5° C. for three days, and then removed, stored, and used.

Specifically, the YPD medium for yeast culture was prepared by mixing carbon sources including 100 g/L of barley, 100 g/L of rice flour, 100 g/L of potato, 20 g/L of glucose, and 2.4 g/L of agar.

The barley, rice flour, and potato were washed, peeled, and cut into small pieces, and then heated with an appropriate amount of water. Thereafter, a nitrogen source was mixed and stirred at 150 rpm, and then sterilized in an autoclave at 121° C. for 20 minutes, and subsequently cooled. When the temperature inside the autoclave decreased to about 50° C., the medium was taken out and placed on a sterilized workbench.

### 1-2. Preparation of Mixed Medium for Bioconversion

For bioconversion, a mixed medium was prepared as follows.

As a carbon source, 20 g/L of maltose was prepared; as a nitrogen source, 5.0 g/L of arginine was prepared; and as a catalytic factor, 0.1 g/L of calcium nitrate tetrahydrate was prepared. These components were mixed with distilled water to achieve the above concentrations, and the mixture was stirred until completely dissolved. The medium was sterilized at 121° C. for 20 minutes, shaken, removed, and then placed in a sterilized inoculation vessel to cool before use.

### 1-3. Bioconversion

The yeast culture broth was added to the sterilized mixed medium for bioconversion at 6% (v/v), sealed with a sterilized filtration membrane, and then cultured in a shaking incubator maintained at 27.5° C. for three days.

Subsequently, ginsenoside Re was added as a precursor substrate at a concentration of 0.75 g/L, and a conversion process was performed for seven days.

Thereafter, the obtained converted product was isolated by a conventional method, and as a result, it was confirmed that the conversion rate was 90% or higher and the molar ratio of the converted product was 60% or higher.

From the above results, it was confirmed that the bioconversion using yeast according to the present disclosure provides a high-efficiency, environmentally friendly, and directed structural modification method, and has the advantage of exhibiting high biological stability and high genetic stability. That is, it was confirmed that the method exhibits high specificity, produces few by-products, and has a clear and concise chemical background, making it useful for mass production, development, and application of the final product. Accordingly, the present disclosure was completed.

As set forth above, the method for synthesizing a metabolite according to an exemplary embodiment of the present disclosure exhibits excellent conversion efficiency by enabling conversion of a precursor substrate into a nitrogen-containing precursor-derived metabolite at a high conversion rate of 90% or higher, and is useful for large-scale production, manufacture, and application.

Although the present disclosure has been described in detail with reference to specific exemplary embodiments, it will be apparent to those skilled in the art that such descriptions are merely preferred exemplary embodiments and do not limit the scope of the present disclosure. Accordingly, the scope of the present disclosure should be defined by the appended claims and equivalents thereof.

### Accession Number

Depositary Institution: Korean Collection for Type Cultures (KCTC), Korea Research Institute of Bioscience BioTechnology (KRIBB)
Accession Number: KCTC16041BP
Date of Deposit: Sep. 6, 2024

What is claimed is:

1. A method for producing a nitrogen-containing metabolite, the method comprising:
culturing Candida intermedia (Accession number: KCTC16041BP) or a mutant strain thereof to obtain a culture broth;
preparing a mixed medium containing one or two or more carbon sources selected from the group consisting of maltose, glucose, poly(xylose), fructose, lactose, sucrose, and soluble starch, and one or two or more nitrogen sources selected from the group consisting of ammonium sulfate, arginine, glutamine, and asparagine; and
adding the culture broth and a precursor substrate to the mixed medium and performing culture.

2. The method of claim 1, wherein the mixed medium further contains one or two or more catalytic factors selected from the group consisting of calcium nitrate tetrahydrate, magnesium nitrate hexahydrate, magnesium sulfate, monopotassium phosphate, and monosodium phosphate.

3. The method of claim 2, wherein the catalytic factor is contained in the mixed medium at a concentration of 0.05 g/L to 0.15 g/L.

4. The method of claim 1, wherein the precursor substrate comprises saponin.

5. The method of claim 4, wherein the saponin comprises one or two or more selected from the group consisting of ginsenoside Re, ginsenoside Rf, ginsenoside Rg1, ginsenoside Rh1, ginsenoside Rg3, ginsenoside Rh2, and ginsenoside Rb2.

6. The method of claim 1, wherein the precursor substrate is contained in the mixed medium at a concentration of 0.5 g/L to 1.5 g/L.

7. The method of claim 1, wherein the carbon source is contained in the mixed medium at a concentration of 15 g/L to 25 g/L.

8. The method of claim 1, wherein the nitrogen source is contained in the mixed medium at a concentration of 2.5 g/L to 7.5 g/L.

9. A method for isolating and purifying a nitrogen-containing metabolite, the method comprising isolating and purifying a transformed culture broth obtained according to the method for producing a nitrogen-containing metabolite of claim 1.

10. The method of claim 9, wherein the isolating and purifying is performed by at least one of the following: centrifugation, extraction, filtration, chromatography, or any combination thereof.
